(19) United States
(12) Patent Application Publication
(10) Pub. No.: US 2006/0081264 A1
(43) Pub. Date: Apr. 20, 2006

(54) Self lubricating condom

(76) Inventor: Alexander Vera, Davie, FL (US)

Correspondence Address: Alexander Vera, 8255 W. Sunrise Blvd. #203, Plantation, FL, 33322, US

(21) Appl. No.: 10/967,686

(22) Filed: Oct. 18, 2004

(51) Int. Cl. A61F 5/37

(52) U.S. Cl. 128/884

(57) ABSTRACT

A self lubricating condom for use in mucosal areas and is provided to enhance sexual performance as well as keeping the condom moist and lubricated during sexual intercourse. The invention can be applied to condoms that are configured to cover the penis as well as the female cervix. The condom contains pockets of lubrication made from latex which wear due to friction and releases lubrication at different intervals due to the wear of the plastic of different thinness and thickness, moisture upon impact with the methacrylic acid co-polymer method or moisture upon impact with the gelatin method.

This application claims priority of U.S. Provisional Application Ser. No. 60/510,494 filed Oct. 14, 2003, which is incorporated herein by reference.

### REFERENCES CITED

U.S. Pat. No. 6,651,667 June 2002 Osterberg 128/844; 128/918

U.S. Pat. No. 6,499,483 August 2001 Abullon 28/844; 128/918

### BACKGROUND OF THE INVENTION

This invention relates generally to prophylactic devices. More specifically, the invention relates to a condom for use by a male, but could be applied to the female condom as well. The invention was created for various reasons. First, condoms itself tend to dry out upon sexual intercourse, whether they are condoms made from animal tissue or elastomeric materials such as latex rubber, urethane polymers and the like. When a condom dries out, it is susceptible to breaking. The breaking of a condom puts the person at risk for receiving sexually transmitted diseases and could cause pregnancy. The condom is generally an application used for contraceptive means and for prevention of sexually transmitted diseases (S.T.D.) and AIDS. The idea of the invention is so the condom does not have to be withdrawn during sexual intercourse for reapplication of lubrication. The condom will be self sufficient in releasing an adequate supply of lubrication onto itself upon entering a mucous membrane. Additionally, this condom is essential to anal sex. As the anal cavity is not a self lubricating orifice, this condom would provide the necessary lubrication to ease the pain of anal intercourse.

### SUMMARY OF THE INVENTION

The present invention is the first self lubricating condom invented. The condom is made of a natural rubber latex sheath and can even be applied to condoms made from animal membrane or tissue, which completely covers the penis with a closely fitted membrane. However, the condom itself should preferably be manufactured of latex rubber. However, it will be appreciated that the condom can also be manufactured from elastomeric materials such as urethane polymers, rubber, synthetic rubber or non-allergic plastic (e.g., surgical plastic). The condom may come in an assortment of different varieties such as, lubricated, non-lubricated, ribbed, thin, studded, different colors, nipple end, etc. The latex condom has rounded bumps all over it that are ‘pockets’ filled with lubrication fluid. Once friction is placed on the condom, the rounded bumps, A.K.A ‘pockets’, are rubbed (due to the friction such as intercourse or rubbing) and the thin plastic covering is worn and lubrication is released onto the condom. Each plastic covering over the pockets filled with lubrication are covered by a thin or thick latex plastic that wears at different intervals thus providing constant lubrication on the condom. The wear is caused at different intervals due to the plastic encasing each pocket filled with lubrication being thin (to allow lubrication to release fast) and some plastic coverings over the lubrication pockets being thicker (to extend the use of sexual intercourse and having lubrication release later), thus keeping the condom lubricated constantly during intercourse. The idea is for the user to not have to withdraw from sexual intercourse to reapply lubrication to the condom. Although the rounded bumps (pockets containing lubrication) are made up of a plastic that wears and breaks to allow the release of lubrication onto the condom, the condom itself is made from a regular standard regulation latex condom that does not pose a risk of breaking or tearing if used properly. The self lubricating condom invention can be applied to the male condom and female condom. The invention works the same way for both male and female condoms. An additional method is to use methacrylic acid co-polymer as the encasement to hold the lubrication fluid. If this method is chosen, any moisture or fluid from the mucous membrane will cause the methacrylic acid co-polymer to gel up and release the lubrication onto the condom. The consistency of the formulation of the methacrylic acid co-polymer will be so that it will release lubrication at different intervals to keep the condom lubricated consistently. Another method to use is to substitute gelatin as encasement for the lubrication pockets. The same procedure would apply once the gelatin pockets filled with lubrication comes in contact with moisture or a mucous membrane. The moisture or fluids from the mucous membrane will cause the gelatin to gel up and release the lubrication onto the condom

### DESCRIPTION OF THE DRAWINGS

In order to facilitate a better understanding of this description and as an integrated part of the same, a series of figures are attached which, as a non-limitative illustration, represent the following.

FIG. 1 shows a perspective view of the condom in rolled-up condition. Notice that number 1 indicates the condom body itself, number 3 indicates the membrane and number 4 indicates the pocket filled with lubrication on the outside of the condom.

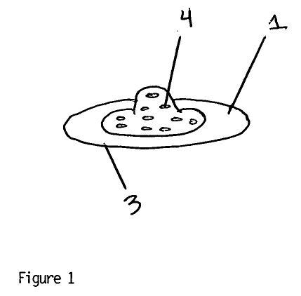

FIG. 2 shows a perspective view of the condom in rolled-up condition again. This time, notice that number 1 indicates the condom body itself, number 2 indicates the pocket filled with lubrication on the inside of the condom and number 4 indicates the pocket filled with lubrication on the outside of the condom.

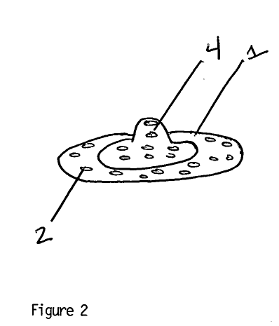

FIG. 3 shows the view of the condom, object of the present invention, unrolled. Number 1 indicates the condom body itself, number 2 indicates the pocket filled with lubrication on the inside of the condom, number 3 indicates the condom membrane and number 4 indicates the pocket filled with lubrication on the outside of the condom.

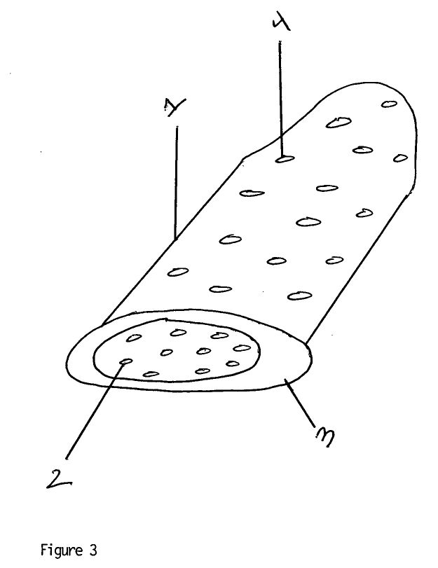

FIG. 4 shows a perspective view of the condom fully unrolled, where the pockets filled with lubrication can clearly be seen throughout the body of the condom. Number 1 indicates the condom body itself, number 3 indicates the condom membrane and number 4 indicates the pocket filled with lubrication on the outside of the condom.

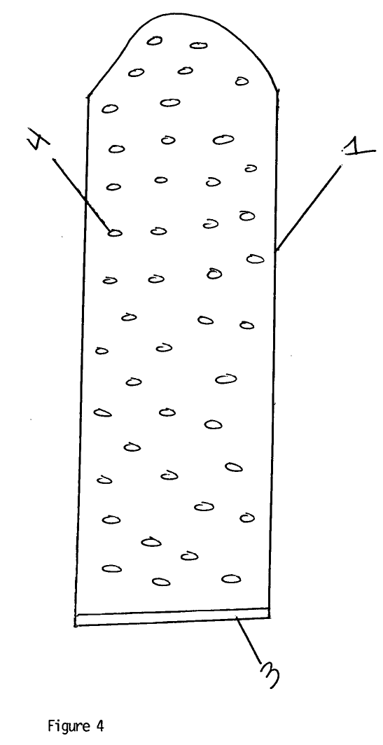

FIG. 5 shows a perspective view of the condom fully unrolled, where the pockets filled with lubrication can clearly be seen breaking and releasing the lubrication they encase throughout the body of the condom. Number 1 indicates the condom body itself, number 3 indicates the condom membrane and number 4 indicates the pocket filled with lubrication on the outside of the condom.

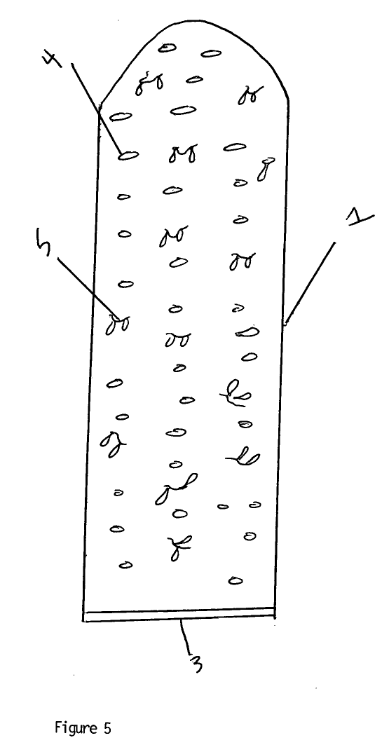

FIG. 6 shows a perspective view of the condom fully unrolled, where the pockets filled with lubrication can clearly be seen having all just about broken open releasing the lubrication they encase throughout the body of the condom. Number 1 indicates the condom body itself, number 3 indicates the condom membrane and number 4 indicates the pocket filled with lubrication on the outside of the condom.

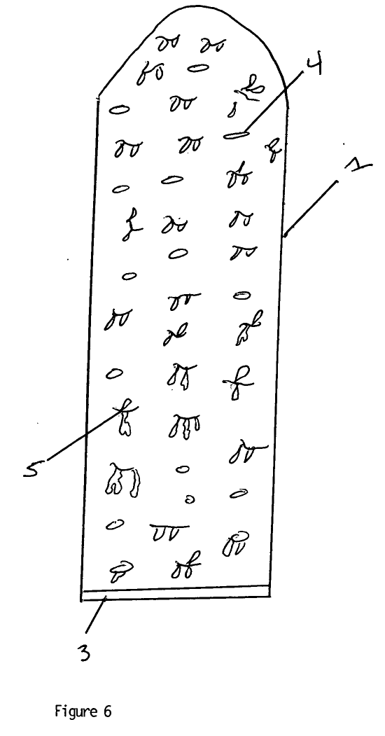

The foregoing drawings, discussion and description are illustrative of particular embodiments of the present invention, but are not meant to be limitations upon the practice thereof. In view of the foregoing, yet other modifications and variations of the present invention will be apparent to one of skill in the art. It is the following claims, including all equivalents, which define the scope of the invention.

1. A condom comprising:
Of natural rubber latex sheath or animal membrane which completely covers the penis with a closely fitted membrane, which contains pockets of lubrication that wear at different intervals keeping the condom moist and lubricated to prevent breakage of condom or pain for their sexual partner due to dryness and chaffing. Another method comprises the use of methacrylic acid co-polymers or gelatin that releases the lubrication it encases upon impact with moisture. This invention can also be applied to the female condom, which is a sheath like material made of polyurethane.

2. The condom of claim 1, wherein said pockets of lubrication have been added to different places on the outside of the condom and can be added to the inside of the condom to disperse lubrication evenly among the condom and sexual organs of user and their partner.

3. The condom of claim 1, further comprising of small circular pockets of lubrication adhered from top to bottom of condom.

4. The condom of claim 3, wherein said pockets filled with lubrication encasements are comprised of thinner layers of plastic and or methacrylic acid co-polymer or gelatin allowing the pockets filled with lubrication to wear or gel and explode lubrication onto condom and sexual organs of user and their partner.

5. The condom of claim 1, further comprising pockets of lubrication positioned on said condom body.

6. The condom of claim 5, wherein said pockets are made with different layers of latex, mostly very thin, that wears at different intervals due to frictions. The latex pocket encasements will be made of latex at different viscosities.
A) By making latex pocket encasements holding lubrication at viscosities containing either 0.10% or 0.30% or 0.45% or 0.60% or 0.70% or 2% or 3% or 7% or 8% or 10% or 13% latex per each pocket encasement, this ensures breakage upon friction at different intervals during intercourse, thus releasing the lubrication it encases onto the condom itself. The viscosity of the condom itself remains as is, unaffected by the breakage. The ingredients used to make the standard latex condom itself remains as well. One percentile of latex will be determined for each latex pocket encasement.
B) Another method is to use a methacrylic acid co-polymer that gels due to moisture and releases lubrication onto condom. The methacrylic acid co-polymer composition will be formulated so there are different amounts of thick or thin pockets encased with lubrication allowing methacrylic acid co-polymer pockets encased with lubrication to wear and release lubrication at different intervals upon impact with moisture or mucous membranes.
C) Another method, the gelatin method, for use with animal membrane condoms consists of material comprising of using gelatin to encase pockets filled with lubrication, composed of vegetable oil, gelatin (can be from pork or from other sources to create gelatin); additional ingredients containing 2% or less of: glycerin, sucralose, mineral oil, carnuba wax and tocopherols. Another method, called the gelatin method, for use with latex condoms consists of material comprising of using gelatin to encase pockets filled with lubrication, composed of gelatin (can be from pork or from other sources to create gelatin); and additional ingredients that contain 2% or less of: glycerin, sucralose, carnuba wax. Gelatin composition will be formulated so there are different amounts of thick or thin pockets encased with lubrication allowing gelatin pockets encased with lubrication to wear and release lubrication at different intervals upon impact with moisture or mucous membranes.

7. The condom of claim 5, wherein said pocket is filled with lubrication.

8. The condom of claim 7, wherein said filler material comprises a fluid (lubrication) which maintains friction on said condom and keeps condom from breaking.

9. The condom of claim 8, wherein said fluid within the pocket encasement (lubrication) is selected from a group consisting of, but not limited to, purified water, glycerin, propylene glycol, cellulose polymer, polyethylene oxide, sodium benzoate, methyl paraben, carbomer 981, diazolidynl urea, EDTA.

10. The condom of claim 7, wherein said fluid (lubrication) material can be but not limited to a formula composed of purified water, glycerin, propylene glycol, cellulose polymer, polyethylene oxide, sodium benzoate, methyl paraben, carbomer 981, diazolidynl urea, EDTA.

11. The condom of claim 5, wherein said plastic covering over pockets filled with lubrication is composed of thin layers of latex that are different in thickness and thinness throughout allowing breakage at different intervals. Another method has the cover of the pockets of lubrication comprised of a methacrylic acid co-polymer or gelatin casing mentioned in claim 6.

12. The condom of claim 1, wherein said pockets filled with lubrication further comprises a smooth outer surface.

14. The condom of claim 1, which can additionally use pockets filled of lubrication made with methacrylic acid co-polymer filled with lubrication to swell up to a gel to lubricate such condom as mentioned in claim 6.

15. The condom of claim 1, wherein substituting methacrylic acid co-polymer for encasing the pockets filled with lubrication comprises of purified water, glycerin and methacrylic acid co-polymer.

16. The condom of claim 1, which can additionally use gelatin as the outer casing for the pockets filled with lubrication in a formulation mentioned in claim 6.

17. A self lubricating condom comprising:
A self lubricating condom for use in mucosal areas and is provided to enhance sexual performance as well as keeping the condom moist and lubricated during sexual intercourse. The invention can be applied to condoms that are configured to cover the penis as well as the female cervix. The condom contains pockets of lubrication made from latex which wear due to friction and releases lubrication at different intervals due to the wear of the plastic of different thinness and thickness, moisture upon impact with the methacrylic acid co-polymer method or moisture upon impact with the gelatin method.

18. The condom of claim 17, wherein said pockets filled with lubrication are small enough not to interfere with such condom usage but may be larger to enhance sexual pleasure if desired.

19. The condom of claim 17, wherein said pockets of lubrication are placed throughout outside, and if desired, inside of condom that disperse lubrication when worn at different intervals onto condom or if using other method of methacrylic acid co-polymer or gelatin, exposes lubrication based on moisture causing release of lubrication.

20. The condom of claim 17, wherein said pockets filled with lubrication are devised of latex plastic, mostly thin, but also of different thickness and gauge to wear at different intervals upon friction caused by sexual intercourse, not to exclude but to include claim to substitute latex with methacrylic acid co-polymer filled with lubrication to disperse evenly onto condom during sexual intercourse as mentioned in claims 4, 6, 11, 14 and 15. Additionally to include the use of gelatin formulation mentioned in claim 6, 11, 16, 17 and 19 as substitution or addition for the fore mentioned.

21. A self lubricating condom comprising:
A self lubricating condom for use in mucosal areas and is provided to enhance sexual performance as well as keeping the condom moist and lubricated during sexual intercourse. The invention can be applied to condoms that are configured to cover the penis as well as the female cervix. The condom contains pockets of lubrication made from latex which wear due to friction and releases lubrication at different intervals due to the wear of the plastic of different thinness and thickness, moisture upon impact with the methacrylic acid co-polymer method or moisture upon impact with the gelatin method.

22. The condom of claim 21, wherein said pockets encased with lubrication are intended to release lubrication at different time intervals during sexual intercourse, thus releasing lubrication consistently and constantly to keep condom from breaking.

23. The condom of claim 21, further explaining that pockets filled with lubrication encased by thin and thick viscosities of latex or methacrylic acid co-polymer or gelatin will release pockets filled with lubrication upon impact with moisture or mucous membrane.

24. The condom of claim 23, wherein said methacrylic acid co-polymer or gelatin will gel upon impact with moisture or mucous membrane thus releasing lubrication onto condom and sexual organs of user and partner if substituted, rather than using the method of using pockets encased with lubrication having an outer containment comprised of latex of different thickness, thinness and gauge, which breaks upon wear and friction.
